(19) United States
(12) Patent Application Publication
(10) Pub. No.: US 2008/0076830 A1
(43) Pub. Date: Mar. 27, 2008

(54) Pharmaceutical Composition in the Form of a Water Soluble Solid Dosage Form

(75) Inventors: Ernest Schay, Edenvale (ZA); Walter Focke, Pretoria (ZA); Lushane Walbrugh, Germiston (ZA)

Correspondence Address: BAYER HEALTHCARE LLC, P.O.BOX 390, SHAWNEE MISSION, KS, 66201, US

(73) Assignee: BAYER HEALTHCARE AG, Leverkusen (DE)

(21) Appl. No.: 11/574,957

(22) PCT Filed: Aug. 27, 2005
(86) PCT No.: PCT/EP05/09265

§ 371 Date: Aug. 27, 2007

(30) Foreign Application Priority Data
Sep. 9, 2004 (ZA) 2004/7206

(51) Int. Cl. A01N 37/52; A01P 13/00; A61K 47/32

(52) U.S. Cl. 514/636; 514/772.4

(57) ABSTRACT

A pharmaceutical composition comprising an active pharmaceutical composition and a water-soluble polymeric composition, the resultant mixture being formed into a solid, water-soluble dosage form is provided.

### FIELD OF THE INVENTION

This invention relates to a pharmaceutical composition and, more particularly, to a pharmaceutical composition in the form of a water-soluble solid dosage form.

### BACKGROUND TO THE INVENTION

In this specification the term “pharmaceutical” shall have its widest meaning and include compounds used in the treatment of humans, animals and plants. This term includes pesticides and in this specification the term “pesticide” is intended to encompass within its scope herbicides and compositions for eradicating or controlling animal pests.

Liquids in general, and pesticides in particular, are usually sold as a liquid concentrate in metal or plastic containers from which a desired quantity of the product is decanted before use.

Pesticides, by their nature, are hazardous chemicals and disposal of empty pesticide containers is a problem as the containers usually contain a pesticide residue which can leach into the environment with disastrous consequences. To an extent, similar problems can be experienced with full or partially full containers, particularly metal containers or plastic containers that have stripped cap screws. Also, containers frequently fall and break during transport.

Another problem with the containers is the use, particularly in developing countries where amenities such as running water are lacking, of empty containers to store water for domestic use. The dangers of such a practise need no elaboration.

One attempt to address the above problem involves supplying the active pesticide ingredient in powder form in plastic bags. A desired quantity of the active is measured out and mixed with water shortly before using. To a large extent the plastic bags, while requiring less storage space than metal or plastic containers, suffer from the same disadvantages as the containers particularly when they are stored in damp environments and also when measuring the correct quantity of the powder.

### OBJECT OF THE INVENTION

It is an object of this invention to provide a pharmaceutical composition and, more particularly, to provide a pharmaceutical composition in the form of a water-soluble solid dosage form which at least partly alleviates the abovementioned disadvantages.

### SUMMARY OF THE INVENTION

In accordance with this invention there is provided a pharmaceutical composition comprising an active pharmaceutical composition and a water-soluble polymeric composition, the resultant mixture being formed into a solid, water-soluble dosage form.

There is further provided for the melting point of the polymeric composition to be lower than the melting point of the active pharmaceutical composition; for the polymeric composition to include a polyethylene glycol; and for the active pharmaceutical composition to be a pesticide.

There is further provided for the pesticide to be a herbicide. Alternatively there is provided for the pesticide to be suitable for controlling or eradicating mammalian pests, preferably ectoparasites and further preferably an acaricide.

There is also provided for the acaricide to be a pesticide belonging to the amidine group of pesticides, preferably AMITRAZ.

There is further provided for the pharmaceutical composition to include at least one suitable dispersant, and/or at least one disintegrant, and/or at least one surfactant, and/or at least one filler; for the dispersant to be a sodium salt of the condensation product of naphthalene sulphuric acid, preferably WETTOL D2; for the disintegrant to be sodium starch glycolate; and for the surfactant to be a non-ionic surfactant belonging to the nonylphenol ethoxylate group, preferably ARKOPAL N090.

Still further according to this aspect of the invention there is provided for the composition to include AMITRAZ, calcium carbonate, ARKOPAL N090, WETTOL D2 and polyethylene glycol 6000; for the constituents to be present in the following ranges (% m/m):

| | | |
|---|---|---|
| AMITRAZ | 20 to 60% | m/m, |
| Calcium carbonate | 5 to 17% | m/m, |
| ARKOPAL N090 | 1 to 4% | m/m, |
| WETTOL D2 | 0.6 to 2% | m/m, and |
| Polyethylene glycol 6000 | 10 to 75% | m/m. |

Yet further according to the invention there is provided for the composition to be constituted as follows:

| | | |
|---|---|---|
| AMITRAZ | 58% | m/m, |
| Calcium carbonate | 15% | m/m, |
| ARKOPAL N090 | 4% | m/m, |
| WETTOL D2 | 2% | m/m, and |
| Polyethylene glycol 6000 | 21% | m/m. |

The invention also provides a method of manufacturing a pharmaceutical composition in solid dosage form which includes melting a water-soluble polymeric composition, adding an active pharmaceutical composition and casting the resultant mixture into moulds.

### BRIEF DESCRIPTION OF ONE EMBODIMENT OF THE INVENTION

The invention will be described below by way of example only and with reference to the accompanying example which is for an acaricidal composition.

### DETAILED DESCRIPTION OF EMBODIMENTS OF THE INVENTION

A pharmaceutical composition according to the invention is produced by melting a polymeric composition, in this embodiment polyethylene glycol (PEG) 6000 at 60° C., adding the acaricide, AMITRAZ milled together with calcium carbonate, and desired dispersants, surfactants and disintegrants to form a suspension. The resultant liquid is cast into solid dosage form shaped moulds and allowed to cool and solidify to produce a solid dosage form that is soluble in water. Each solid dosage form so produced is individually sealed with a plastics or foil material film.

The following formulation was used:

| CONSTITUENT | % m/m |
|---|---|
| AMITRAZ | 58 |
| CaCO₃ | 15 |
| ARKOPAL N090 | 4 |
| WETTOL D2 | 2 |
| Polyethylene glycol (PEG) 6000 | 21 |

The solid dosage form has been found to take about 2 hours to dissolve in about 10 litres of water. This makes it highly suitable for applications where sustained release is required.

The concentration of AMITRAZ was standard and based on an effective concentration to control or eradicate acarinids when the solid dosage form produced was dissolved in 10 litres of water.

It is envisaged that the solid dosage forms can be used as a viable alternative to liquid and powder concentrates thus, at least partly, alleviating some of the disadvantages associated with the use of the liquid and powder. It is envisaged that a number of solid dosage forms can be added to any suitable volume of water to make up enough pesticide for application to one animal, in which case one solid dosage form of the pesticide can be made up in a bucket and sponged or sprayed onto the animal, or to several animals, in which case several solid dosage forms of the pesticide can be dissolved in a dip tank or in a spray race reservoir. Alternatively, instead of using several smaller solid dosage forms, one large (2-5 kg) dosage form could be produced and packed as described above.

Using a water soluble polymeric composition as a carrier in the manner described has the further advantage that it is easy to vary solid dosage form sizes. The mould is simply changed to a suitable size whereas with normal tableting equipment a change in tablet size generally requires expensive changes to dies and presses.

Also, by varying the amount of active ingredient and excipients in a formulation it is possible to control the dissolution rate of the solid dosage form. For example, increasing the concentration of AMITRAZ causes a slowing of the rate of dissolution. This makes it possible to provide a sustained release formulation where required, thus increasing the dissolution time from minutes to hours or days.

It will be appreciated that a wide variety of pesticides and additives can be used in the above invention without departing from the scope thereof. In particular, the pesticide active ingredient can be an acaricide as described or the pesticide may target other ectoparasites. Alternatively the pesticide active ingredient may target a variety of endoparasites such as nematodes and cystodes to name but two. Where the target is an endoparasite it is envisaged that the solid dosage form can be dissolved in water in a reservoir for an oral dosing gun.

Importantly, any suitable water-soluble polymeric composition can be used. It should, however, preferably have a melting point above that of the pharmaceutical composition with which it is used. Also, where PEG is used it can be of any suitable molecular weight provided it is a solid at room temperature.

In addition to the above, the pesticide active ingredient may also be a herbicide and the solid dosage form dissolved in water in a suitable reservoir shortly before it is applied. Additionally, the active ingredient may also be a protein or disinfectant, or any other pharmacologically active material.

1. A pharmaceutical composition comprising an active pharmaceutical composition and a water-soluble polymeric composition, the resultant mixture being formed into a solid, water-soluble dosage form.

2. A pharmaceutical composition as claimed in claim 1 in which the melting point of the polymeric composition is lower than the melting point of the pharmaceutical composition.

3. A pharmaceutical composition as claimed in claim 1 or claim 2 in which the polymeric composition is a polyethylene glycol.

4. A pharmaceutical composition as claimed in claim 3 in which the polyethylene glycol has a molecular weight selected such that it is a solid at room temperature.

5. A pharmaceutical composition as claimed in claim 3 or claim 4 in which the polyethylene glycol is present in the range 10 to 75 % m/m.

6. A pharmaceutical composition as claimed in any one of the preceding claims in which the active pharmaceutical composition is a pesticide

7. A pharmaceutical composition as claimed in claim 6 in which the pesticide is a herbicide.

8. A pharmaceutical composition as claimed in claim 6 in which the pesticide is suitable for controlling or eradicating mammalian pests.

9. A pharmaceutical composition as claimed in claim 8 in which the pesticide is suitable for controlling or eradicating ectoparasites.

10. A pharmaceutical composition as claimed in claim 8 in which the pesticide is an acaricide.

11. A pharmaceutical composition as claimed in claim 10 in which the acaricide belongs to the amidine group of pesticides.

12. A pharmaceutical composition as claimed in claim 11 in which the pesticide is AMITRAZ.

13. A pharmaceutical composition as claimed in claim 12 in which the AMITRAZ is present in the range 20 to 60 % m/m.

14. A pharmaceutical composition as claimed in any one of the preceding claims which further includes any one or more of at least one dispersant, at least one disintegrant, at least one surfactant and at least one filler.

15. A pharmaceutical composition as claimed in claim 14 in which the dispersant is a sodium salt of the condensation product of naphalene sulphuric acid.

16. A pharmaceutical composition as claimed in claim 15 in which the dispersant is WETTOL D2.

17. A pharmaceutical composition as claimed in any one of claims 14 to 16 in which the dispersant is present in the range 0.6 to 2 % m/m.

18. A pharmaceutical composition as claimed in any one of claims 14 to 17 in which the surfactant is a non-ionic surfactant belonging to the nonylphenol ethoxylate group.

19. A pharmaceutical composition as claimed in claim 18 in which the surfactant is ARKOPAL N090.

20. A pharmaceutical composition as claimed in any one of claims 14 to 19 in which the dispersant is present in the range 1 to 4 % m/m.

21. A pharmaceutical composition as claimed in any one of claims 14 to 20 in which the disintegrant is sodium starch glycolate.

22. A pharmaceutical composition as claimed in any one of claims 14 to 21 which includes calcium carbonate present in the range 5 to 17 % m/m.

23. A method of manufacturing a pharmaceutical composition in solid dosage form which includes melting a water-soluble polymeric composition, adding an active pharmaceutical composition and casting the resultant mixture into moulds.

24. A method as claimed in claim 23 which further includes adding any one or more of at least one dispersant, at least one disintegrant, and at least one surfactant to the melted polymeric composition.

25. A method as claimed in claim 23 or claim 24 in which the active pharmaceutical composition is a pesticide.

26. A method as claimed in claim 25 in which the pesticide is a herbicide.

27. A method as claimed in claim 25 in which the pesticide is suitable for controlling or eradicating mammalian pests.

28. A method as claimed in claim 27 in which the pesticide is suitable for controlling or eradicating ectoparasites.

29. A method as claimed in claim 27 in which the pesticide is an acaricide.

30. A method as claimed in claim 29 in which the acaricide belongs to the amidine group of pesticides.

31. A method as claimed in claim 30 in which the pesticide is AMITRAZ.
